(19) United States
(12) Patent Application Publication
(10) Pub. No.: US 2026/0115179 A1
(43) Pub. Date: Apr. 30, 2026

(54) USE OF RECA INHIBITOR AND DERIVATIVE COMPOUNDS FOR CANCER TREATMENT

(71) Applicant: United Arab Emirates University, Al Ain (AE)

(72) Inventors: Ahmed H. ALMARZOOQI, Al Ain (AE); Alya A. ARABI, Al Ain (AE); Amira BEKDASH, Al Ain (AE); Jisha CHALISSERY, Al Ain (AE); Tiyyaba FURQAN, Al Ain (AE)

(21) Appl. No.: 18/925,790

(22) Filed: Oct. 24, 2024

(51) Int. Cl. A61K 31/4545; A61K 45/06; A61P 35/00

(52) U.S. Cl. CPC A61K 31/4545; A61K 45/06; A61P 35/00

(57) ABSTRACT

The present disclosure provides a use of compound of Formula (I) or (II) for treatment of cancer. More particularly, the present disclosure provides a method of treating or preventing cancer by administering a therapeutically effective amount of a compound of Formula (I) or (II) to a subject in need thereof. More particularly, the present disclosure provides a compound of Formula (I) or (II) or a composition thereof for treatment of breast cancer by inhibiting the growth and/or viability of breast cancer cells.

### TECHNICAL FIELD

The present disclosure relates to methods for treating cancer. In particular, the compounds of Formula (I) or (II) are employed for the treatment of cancer by inhibiting the growth and/or viability of cancer cells.

### BACKGROUND

Cancer is a major health problem and is a leading cause of death worldwide [1]. In 2020, nearly 19.3 million new cases of cancer were reported worldwide, and breast cancer was ranked at the top with 11.7% of cases [2]. The number of new cancer cases is estimated to increase to 29.5 million new cases per year and the number of cancer-related deaths is estimated to increase to 16.4 million per year in 2040. Among females, breast cancer accounts for 36.1% of all reported malignant cancer cases [3]. Chemotherapy can extend survival in patients diagnosed with a wide range of malignancies. However, toxic side-effects to normal cells and tissues limits chemotherapy dose intensity, frequency, and efficacy. For instance, the cardiotoxicity and nephrotoxicity associated with the widely prescribed anti-cancer drugs limit their full therapeutic potential. Consequently, developing strategies to reduce these adverse effects while maintaining or improving the efficacy of the treatment is a key goal in advancing cancer therapy. Despite years of research into the development of new methods of treatment, many types of cancer, including, breast cancer, remain quite common. A safe and effective cancer treatment has been the goal of investigators for a substantial period of time. Successful cancer therapies must be capable of effectively targeting, eliminating, destroying, killing, and/or inhibiting the growth and/or viability of cancer cells, while avoiding severe side-effects. Therefore, there is an urgent need for an effective and safe approach for treating cancer, in particular breast cancer.

### SUMMARY

In accordance with the present invention, disclosed herein is a method of treating or preventing cancer in a subject in need thereof, comprising administering to the subject:
(a) a therapeutically effective amount of a compound of Formula (I):

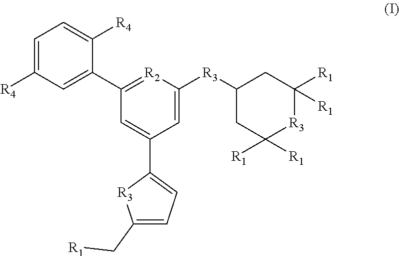

wherein R₁ is independently selected from the group consisting of:
(I) hydrogen
(II) halogen
(III) alkyl or cycloalkyl;
(IV) alkenyl or cycloalkenyl;
(V) alkynyl or cycloalkynyl;
(VI) haloalkyl;
(VII) alkoxy;
(VIII) alkylamino; or
   wherein R₂ is independently selected from the group consisting of:
(IX) a —N;
(X) a —P;
(XI) a —CR, wherein R is selected from the group comprising a hydrogen, an alkyl, a cycloalkyl, a haloalkyl, an alkylamino, or an alkoxy; or
   wherein R₃ is independently selected from the group consisting of:
(XII) an —O;
(XIII) a —S;
(XIV) a —C(R)₂, wherein R is selected from the group comprising a hydrogen, an alkyl, a cycloalkyl, a haloalkyl, an alkylamino, and/or an alkoxy;
(XV) a —NR, wherein R is selected from the group comprising a hydrogen, an alkyl, a cycloalkyl, or a haloalkyl;
(XVI) a —PR, wherein R is selected from the group comprising a hydrogen, a halogen, an alkyl, a cycloalkyl, or a haloalkyl; or
   wherein R₄ is independently selected from the group consisting of:
(XVII) hydrogen;
(XVIII) halogen
(XIX) alkyl or cycloalkyl;
(XX) alkenyl or cycloalkenyl;
(XXI) alkynyl or cycloalkynyl;
(XXII) haloalkyl;
(XXIII) aryl;
(XXIV) cyano;
(XXV) amino;
(XXVI) a —COOR, wherein R is a hydrogen or an alkyl;
(XXVII) a —OCOR, wherein R is an alkyl group;
(XXVIII) acyl;
(XXIX) sulfonyl;
(XXX) a —CON(R)₂, wherein R is selected from the group comprising a hydrogen and/or an alkyl;
(XXXI) a —CONHR, wherein R is selected from the group comprising a hydrogen or an alkyl;
(XXXII) a —PO(OR)₂, wherein R is selected from the group comprising a hydrogen and/or an alkyl;
(XXXIII) an —OR, wherein R is selected from the group comprising a hydrogen, an alkyl, a cycloalkyl, or a haloalkyl;
(XXXIV) an —N(R)₂, wherein R is selected from the group comprising a hydrogen, an alkyl, a cycloalkyl, and/or a haloalkyl;
(XXXV) a —SR, wherein R is selected from the group comprising a hydrogen, an alkyl, a cycloalkyl, or a haloalkyl;
(XXXVI) a nitro;
(XXXVII) a phosphoryl; or
   a pharmaceutically acceptable salt, co-crystal, polymorph, hydrate, solvate, prodrug, N-oxide, bioisostere, or a stereoisomer thereof, or
(b) a therapeutically effective amount of a composition comprising a therapeutically effective amount of the compound of Formula (II):

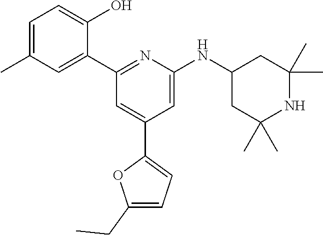

or a pharmaceutically acceptable salt, co-crystal, polymorph, hydrate, solvate, prodrug, or a stereoisomer thereof.

In accordance with the present invention, in a second aspect disclosed herein is a method of treating or preventing cancer in a subject in need thereof, comprising administering to the subject:
(i) a therapeutically effective amount of a compound of Formula (II):

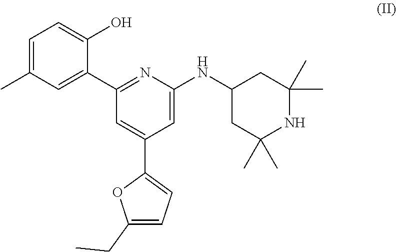

(ii) or a pharmaceutically acceptable salt, co-crystal, polymorph, hydrate, solvate, prodrug, N-oxide, bioisostere or a stereoisomer thereof, or
(ii) a composition comprising a therapeutically effective amount of the compound of Formula (II), or a pharmaceutically acceptable salt, co-crystal, polymorph, hydrate, solvate, prodrug, or a stereoisomer thereof as an active ingredient.

In a preferred embodiment, the present disclosure provides a use of a compound of Formula (I) or (II), or a pharmaceutically acceptable salt, co-crystal, polymorph, hydrate, solvate, prodrug, N-oxide, bioisostere or a stereoisomer thereof, or a composition comprising a therapeutically effective amount of the compound of Formula (I) or (II), or a pharmaceutically acceptable salt, co-crystal, polymorph, hydrate, solvate, prodrug, or a stereoisomer thereof, for the treatment of cancer.

In another aspect, the present disclosure provides a use of a compound of Formula (I) or (II) or a pharmaceutically acceptable salt, co-crystal, polymorph, hydrate, solvate, prodrug, N-oxide, bioisostere or a stereoisomer thereof, or a composition comprising a therapeutically effective amount of the compound of Formula (I) or (II), or a pharmaceutically acceptable salt, co-crystal, polymorph, hydrate, solvate, prodrug, or a stereoisomer thereof, in the manufacture of a medicament for treating cancer.

In yet another aspect, provided herein are methods of treating cancer, which optionally include administering the compounds in conjunction with a chemotherapeutic agent to a subject. A chemotherapeutic agent comprises any treatment applicable for cancer treatment.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1A and 1B illustrate the cancer cell viability in the presence and absence of the compound of Formula (II), measured using MCF7 cell line. Formula (II) is cytotoxic to cancer cells.

FIGS. 2A and 2B illustrate the cancer cell viability in presence and absence of the compound of Formula (II), measured using MDA-MB-231 cell line. Formula (II) is cytotoxic to cancer cells.

### DEFINITIONS

As used in this description and the accompanying claims, the following terms shall have the meanings indicated, unless the context otherwise requires:

As used herein, the singular forms “a”, “an”, and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variation thereof) “comprising”, “comprises”, “comprise”, “consisting essentially of”, “consisting”, and “consists” can be used interchangeably.

The term “exemplary” is used herein to mean “serving as an example, instance, or illustration.” Any embodiment described herein as “exemplary” is not necessarily to be construed as preferred or advantageous over other embodiments.

The term “bioisostere” refers to a compound or group that possesses near molecular shapes and volumes, approximately the same distribution of electrons and which exhibit similar physical properties such as hydrophobicity. Bioisostereic compounds affect the same biochemically associated systems as agonist or antagonists and thereby produce biological properties that are related to each other.

The terms “stereoisomer” or “stereoisomers” refer to any enantiomers, diastereomers or geometrical isomers of the compounds of Formula (I) or (II), wherever they are chiral. When the compounds of the Formula (I) or (II) and related formulae are chiral, they can exist in racemic or in optically active form. It should be understood that the invention encompasses all stereochemical isomeric forms, including diastereomeric, enantiomeric and epimeric forms, and mixtures thereof.

As used herein, the term “composition” is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

As used herein, the terms “comprises” or “comprising” are generally used in the sense of include, that is to say permitting the presence of one or more features or components.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as the event or circumstance where the alkyl is not substituted.

The term “pharmaceutically acceptable” means that, which is useful in preparing a pharmaceutical composition that is generally safe, non-toxic and neither biologically nor otherwise undesirable and includes that which is acceptable for veterinary as well as human pharmaceutical use. These “pharmaceutically acceptable” substances are suitable for use in interact with bodily tissues without causing excessive irritation, allergic responses, or other complications, commensurate with a reasonable benefit/risk ratio.

The term “pharmaceutically acceptable salt” refers to the salts of the compounds, that is pharmaceutically acceptable and that possesses the desired pharmacological activity of the parent compound. The pharmaceutically acceptable salts of the compounds of this disclosure include those derived from suitable inorganic and organic acids and bases. Such salts include: salts derived from inorganic bases such as Li, Na, K, Ca, Mg, Fe, Cu, Zn, Al, Mn, comprising their different oxidation states; salts of organic bases such as N,N′-diacetylethylenediamine, 2-dimethylaminoethanol, isopropylamine, morpholine, piperazine, piperidine, procaine, diethylamine, triethylamine, trimethylamine, tripropylamine, tromethamine, choline hydroxide, dicyclohexylamine, metformin, benzylamine, phenylethylamine, dialkylamine, trialkylamine, thiamine, aminopyrimidine, aminopyridine, purine, pyrimidine, spermidine, and the like; chiral bases like alkylphenylamine, glycinol, phenyl glycinol and the like, salts of natural amino acids such as glycine, alanine, valine, leucine, isoleucine, lysine, arginine, serine, threonine, phenylalanine; unnatural amino acids such as D-isomers or substituted amino acids; salts of acidic amino acids such as aspartic acid, glutamic acid; guanidine, substituted guanidine wherein the substituents are selected from nitro, amino, alkyl, alkenyl, alkynyl, ammonium or substituted ammonium salts. Salts may include acid addition salts where appropriate which are sulphates, nitrates, phosphates, perchlorates, borates, hydrohalides, acetates, tartrates, maleates, citrates, succinates, methanesulfonates, benzoates, salicylates, hydroxynaphthoates, benzenesulfonates, ascorbates, and the likes thereof.

The term “polymorph” refers to the crystallization of the same compound, which differs only in the lattice.

As used herein, the terms “prevents”, “preventing”, “prevention”, “arresting”, “blocking”, “alleviating”, “relieving”, or “inhibits” refer to a method of preventing or inhibiting the onset of a disease and/or its attendant symptoms or barring a subject from acquiring a disease. As used herein, “prevent”, “preventing”, “prevention”, “arresting”, “blocking”, “alleviating”, “relieving”, or “inhibiting” also include delaying the onset of a disease and/or its attendant symptoms and reducing a subject's risk of acquiring a disease.

The terms “solvate” or “solvates” refer to a complex in which the compounds of the Formula (II) of the present disclosure are a proportional amount of solvent molecules combined. A specific solvate in which the solvent is water is referred to as a hydrate.

The term “subject” in accordance with the present invention, includes, e.g., mammals, such as dogs, cats, horses, rats, mice, monkeys, and humans.

The terms “therapeutically effective amount” or “effective amount” refer to the quantity of the compound of Formula (I) or (II) disclosed herein that (i) treats or prevents a specific disease, disorder, or condition or syndrome; (ii) reduces, improves, or eliminates one or more symptoms associated with the disease, disorder, or syndrome; or (iii) delays the onset of one or more symptoms of the disease, disorder, or syndrome described herein. In cases of cancer, the therapeutically effective amount of the drug may reduce cancer cell proliferation, shrink tumor size, inhibit cancer cell migration to peripheral organs, suppress tumor metastasis, slow tumor growth to some extent, and/or alleviate one or more symptoms associated with cancer.

The term “prodrug” as used herein encompasses compounds that, under physiological conditions, are converted into therapeutically active agents. A common method for making a prodrug is to include selected moieties that are hydrolyzed under physiological conditions to reveal the desired molecule. In other embodiments, the prodrug is converted by an enzymatic activity of the host animal. In an embodiment, a prodrug has less than 10 percent activity relative to the free or active drug derived or released therefrom. In an embodiment, a prodrug has less than 5 percent activity relative to the free or active drug derived or released therefrom. In an embodiment, a prodrug has less than 1 percent activity relative to the free or active drug derived or released therefrom.

The terms “—CR”, “—NR”, “—PR”, “—OR”, “—SR”, or “—C(R₂)”, “—N(R)2”, “—COOR”, “—OCOR”, “—CON(R)2”, “—CONHR”, or “—PO(OR)2 refer to one or two R groups attached to a carbon, nitrogen phosphorus, oxygen, or sulfur atom, for example “—CON(R)2” refers to two R group attached to a Nitrogen, as shown in the formula below.

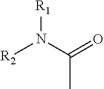

The terms “—S”, “—N”, “—O”, “—P” refer to a sulfur, nitrogen, oxygen, or phosphorus atom, and may include the forms “—S—”, “—N—”, “—O—”, “—P—” where applicable due to formula structure requirements.

The recitation of a listing of chemical groups in any definition of a variable herein includes definitions of that variable as any single group or combinations of listed groups. The recitation of an embodiment for a variable or aspect herein includes that embodiment as any single embodiment or in combination with any other embodiments or portions thereof.

Unless defined otherwise, all technical and scientific terms used herein have the same meaning as commonly understood by one of ordinary skill in the art to which the disclosure belongs. Although any methods and materials like or equivalent to those described herein may be used in the practice or testing of the present disclosure, the preferred materials and methods are described below.

### DETAILED DESCRIPTION

The present invention pertains to a method of treating cancer in a subject in need thereof, comprising a compound of Formula (I), structurally represented as follows:

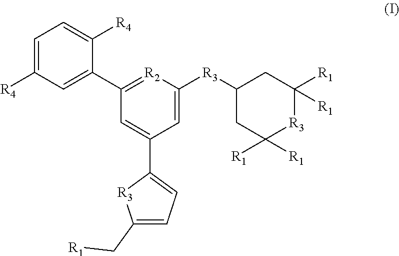

wherein R₁ is independently selected from the group consisting of:
(I) hydrogen
(II) halogen
(III) alkyl or cycloalkyl;
(IV) alkenyl or cycloalkenyl;
(V) alkynyl or cycloalkynyl;
(VI) haloalkyl;
(VII) alkoxy;
(VIII) alkylamino; or
   wherein R₂ is independently selected from the group consisting of:
(IX) a —N;
(X) a —P;
(XI) a —CR, wherein R is selected from the group comprising a hydrogen, an alkyl, a cycloalkyl, a haloalkyl, an alkylamino, or an alkoxy; or
   wherein R₃ is independently selected from the group consisting of:
(XII) an —O;
(XIII) a —S;
(XIV) a —C(R)₂, wherein R is selected from the group comprising a hydrogen, an alkyl, a cycloalkyl, a haloalkyl, an alkylamino, and/or an alkoxy;
(XV) a —NR, wherein R is selected from the group comprising a hydrogen, an alkyl, a cycloalkyl, or a haloalkyl;
(XVI) a —PR, wherein R is selected from the group comprising a hydrogen, a halogen, an alkyl, a cycloalkyl, or a haloalkyl; or
   wherein R₄ is independently selected from the group consisting of:
(XVII) hydrogen;
(XVIII) halogen
(XIX) alkyl or cycloalkyl;
(XX) alkenyl or cycloalkenyl;
(XXI) alkynyl or cycloalkynyl;
(XXII) haloalkyl;
(XXIII) aryl;
(XXIV) cyano;
(XXV) amino;
(XXVI) a —COOR, wherein R is a hydrogen or an alkyl;
(XXVII) a —OCOR, wherein R is an alkyl group;
(XXVIII) acyl;
(XXIX) sulfonyl;
(XXX) a —CON(R)₂, wherein R is selected from the group comprising a hydrogen and/or an alkyl;
(XXXI) a —CONHR, wherein R is selected from the group comprising a hydrogen or an alkyl;
(XXXII) a —PO(OR)₂, wherein R is selected from the group comprising a hydrogen and/or an alkyl;
(XXXIII) an —OR, wherein R is selected from the group comprising a hydrogen, an alkyl, a cycloalkyl, or a haloalkyl;
(XXXIV) an —N(R)₂, wherein R is selected from the group comprising a hydrogen, an alkyl, a cycloalkyl, and/or a haloalkyl;
(XXXV) a —SR, wherein R is selected from the group comprising a hydrogen, an alkyl, a cycloalkyl, or a haloalkyl;
(XXXVI) a nitro;
(XXXVII) a phosphoryl; or
   a pharmaceutically acceptable salt, co-crystal, polymorph, hydrate, solvate, prodrug, N-oxide, bioisostere, or a stereoisomer thereof, or
(b) a therapeutically effective amount of a composition comprising a therapeutically effective amount of the compound of Formula (II):

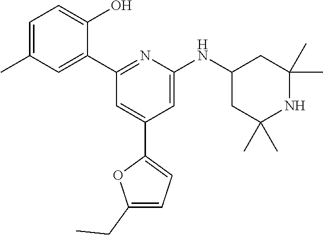

or a pharmaceutically acceptable salt, co-crystal, polymorph, hydrate, solvate, prodrug, or a stereoisomer thereof.

In a preferred embodiment, the present invention pertains to a method of treating cancer in a subject in need thereof, comprising a compound of Formula (II), structurally represented as follows:

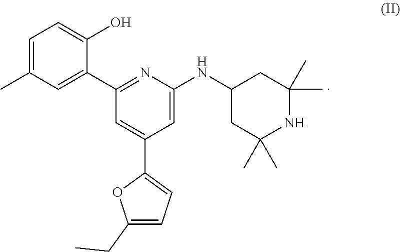

The compound of Formula (II) is conventionally known as a RecA inhibitor, but the use of the compound in treatment of cancer was previously unknown. We identified that the compound of Formula (II) controls cancer by inhibiting the growth and/or viability of cancer cells. In addition, the compound has an inhibitory effect that is similar or more effective than that of agents previously utilized in cancer treatment, such as cisplatin (FDA-approved for cancer treatment) and artesunate, and has a better activity profile compared to primaquine and cycloguanil.

The compounds of Formula (II) may be used in the form of a free base, a salt, a co-crystal, a polymorph, a hydrate, a solvate, a prodrug, a bioisostere, or a stereoisomer thereof, including pharmaceutically acceptable forms. In certain embodiments, the compound of Formula (II) may be a pharmaceutically acceptable salt.

In one aspect, the present invention discloses a method of treating or preventing cancer in a subject in need thereof, comprising administering to the subject a therapeutically effective amount of a composition comprising the compound of Formula (I) or (II), or a pharmaceutically acceptable salt, co-crystal, polymorph, hydrate, solvate, prodrug, N-oxide, bioisostere or a stereoisomer thereof, and one or more excipients and/or carriers.

In another aspect, disclosed herein is the use of a compound of Formula (II):

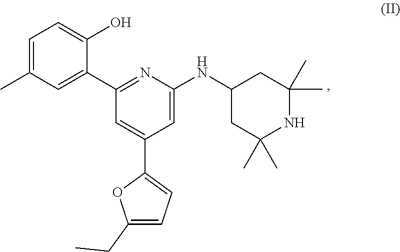

or a pharmaceutically acceptable salt, co-crystal, polymorph, hydrate, solvate, prodrug, N-oxide, bioisostere or a stereoisomer thereof, in the treatment of cancer.

In yet another aspect, the present disclosure provides a composition comprising the compound of Formula (I) or (II), or a pharmaceutically acceptable salt, co-crystal, polymorph, hydrate, solvate, prodrug, or a stereoisomer thereof, for use in the treatment of cancer.

In an embodiment, the cancer includes, but not limited to: Adenocarcinoma of the Colon; Adenocarcinoma of the Esophagus; Adenocarcinoma of the Lung; Adenocarcinoma of the Prostate; Adenocarcinoma of the Rectum; Advanced Adult Primary Liver Cancer; Advanced Non-Nasopharyngeal Head and Neck Carcinoma; Anaplastic Astrocytoma; Anaplastic Oligodendroglinm; Anaplastic Thyroid Cancer; Bladder Cancer: Brain Tumor: Breast Cancer; Breast Cancer in Situ; Breast Neoplasms; Bronchoalveolar Cell Lung Cancer; Cancer of the Fallopian Tube; Carcinoma, Squamous Cell; Cervix Neoplasms; Colon Cancer; Colorectal Cancer; Epithelial Mesothelioma; Esophageal Cancer: Esophagogastric Cancer; Follicular Thyroid Cancer; Gastric Cancer; Gastrinoma; Gastrointestinal Carcinoid, Giant Cell Glioblastoma; Glioblastoma; Glioblastoma Multiforme; Head and Neck Cancer; Hepatocellular Carcinoma; Hypopharyngeal Cancer; Inoperable Locally Advanced Squamous Cell Carcinoma of Head and Neck; Insulinona; Intraductal Breast Carcinoma; islet Cell Carcinoma; Large Cell Lung Cancer: Laryngeal Cancer; Lip and Oral Cavity Cancer; Lip Cancer; Liver Cancer; Lung Adenocarcinoma With Bronchiole-Alveolar Feature; Lung Cancer; Male Breast Cancer; Medullary Thyrod Cancer; Meningeal Tumors; Metastatic Colorectal Cancer; Metastatic Gastrointestinal Carcinoid Tuinor, Metastatic Pancreatic Carcinoma; Mixed Glioimas; Myelogenous Leukemia, Acute; Nasopharyngeal Carcinoma; NeuroNastoma; Non-Metastatic (T2T4, N0-N3, MO: Stages II and III) and Histologically-Confirmed Intestinal GC; Non-Metastatic Prostate Cancer; Nonresectable Adrenocortical Carcinoma; Non-Small Cell Lung Cancer; Nose Cancer; Oligodendroglial Tumors; Oral Cancer; Oropharyngeal Cancer; Osteosarcoma; Ovarian Cancer: Ovarian Neoplasms; Pancreatic Cancer: Papillary Thyroid Cancer; Peritoneal Carcionma; Pharynx Cancer; Pneumonic-Type Adenocarcinoina (P-ADC); Primary Hepatocellular Carcinoma; Prostate Cancer: Rectal Cancer; Recurrent Adult Primary Liver Cancer; Recurrent Breast Cancer; Recurrent Colon Cancer; Recurrent Endometrial Cancer, Recurrent Esophageal Cancer; Recurrent Glioblastonna: Recurrent Rectal Cancer: Recurrent Skin Cancer: Refractory Germ Cell Tumors Expressing EGRF; Renal Cell Cancer: Rhabdonyosarcomas; Sarcomatous Mesothelioma; Skin Cancer; Soft Tissue Sarcoma, Squamous Cell Carcinoma of the Esophagus; Squamous Cell Carcinoma of the Head and Neck; Squamous Cell Carcinoma of the Skin, Squamous Cell Lung Cancer; Stage II Esophageal Cancer; Stage III Esophageal Cancer, Synovial Sarcoma; Thorax and Respiratory Cancer; Throat Cancer; Thyroid Cancer; Transitional Cell Cancer of the Renal Pelvis and Ureter; Transitional Cell Carcinoma of the Bladder: Tubal Carcinoma; Unspecified Childhood Solid Tumor; Untreated Childhood Brain Stem Glioma; Urethral Cancer.

In a preferred embodiment the cancer is breast cancer.

In an embodiment, the composition further comprises a pharmaceutically acceptable carrier or excipient.

In any of the preceding embodiments, the method of treating or preventing cancer comprises optionally administering an additional therapeutic agent.

In one embodiment, the additional therapeutic agent is administered prior to, concurrently with, or subsequently to the administration of the compound of Formula (I) or (II) or the composition comprising the compound of Formula (I) or (II). In another embodiment, the additional therapeutic agent is administered prior to the administration of the compound of Formula (I) or (II) or the composition comprising the compound of Formula (I) or (II). In a further embodiment, the additional therapeutic agent is administered concurrently with the administration of the compound of Formula (I) or (II) or the composition comprising the compound of Formula (I) or (II). In yet another embodiment, the additional therapeutic agent is administered subsequent to the administration of the compound of Formula (I) or (II) or the composition comprising the compound of Formula (I) or (II).

In an embodiment, the compound of Formula (I) or (II) exhibits therapeutic effect by targeting blocking the growth and/or viability of cancer such as breast cancer. In an embodiment, it has a similar or more inhibitory effect compared to agents that have been studied for their anti-cancer effects. In an embodiment, the compound of Formula (I) or (II) has a similar inhibitory effect compared to cisplatin (FDA-approved for cancer treatment) and artesunate, and a better activity profile compared to primaquine and cycloguanil. In an embodiment, the compound of Formula (II) exhibits an IC₅₀in MCF7 cells (5.90±0.29 μM, see Table 1), comparable to that of cisplatin (5.75±0.02 μM over 24 h treatment). However, the compound exhibits a better toxicity profile compared to cisplatin. In addition, at concentrations higher than IC₅₀(i.e., 10 μM), the compound of Formula (II) is more advantageous and can kill almost 96-99.7% of cancer cells, while cisplatin at the same concentration leaves around 30% of viable cells in culture. Cisplatin thus kills around 70% of the cancer cells only [4].

Thus, in an embodiment, the compounds of the present invention kill about more than 70% of cancer cells. In an embodiment, the compounds of the present invention kills about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%, of cancer cells.

In an embodiment, compound (II) has a better activity profile compared to primaquine and cycloguanil. The IC₅₀ of cycloguanil and primaquine in MCF-7 cancer cell line was 20.30±0.08 μM and 29.90±0.02 μM, respectively, following 48 hours of treatment [5]. However, the IC₅₀ of Formula (II) in MCF-7 after 48 hours was 4.41±0.90 μM. Hence, Formula (II) presents a higher cytotoxic ability and, thus, better activity profile compared to cycloguanil and primaquine.

The compositions and methods of the present disclosure are utilized to treat a subject in need thereof. In certain embodiments, the subject is a mammal such as a human or a non-human mammal. When administered to an animal, such as a human, the composition or the compound is preferably administered as a pharmaceutical composition comprising, for example, the compounds of the present invention and a pharmaceutically acceptable carrier. Pharmaceutically acceptable carriers are well known in the art and include, for example, aqueous solutions such as water or physiologically buffered saline or other solvents or vehicles such as glycols, glycerol, and oils such as olive oil or injectable organic esters. In a preferred embodiment, when such pharmaceutical compositions are for human administration, particularly for invasive routes of administration (i.e., routes, such as injection or implantation, that circumvent transport or diffusion through an epithelial barrier), the aqueous solution is pyrogen-free or substantially pyrogen-free. The phrase “pharmaceutically acceptable carrier” as used herein means a pharmaceutically acceptable material, composition, or vehicle, such as a liquid or solid filler, diluent, excipient, solvent, or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The excipients can be chosen, for example, to effect delayed release of an agent or to selectively target one or more cells, tissues, or organs.

The pharmaceutical composition may be formulated as tablets, pills, powders, granules, capsules, suspensions, liquids for internal use, emulsions, syrups, aerosols, or sterile injectable solutions. Depending on the intended use, the composition can be administered to a subject by any of a number of routes of administration including, for example orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin or as an eye drop). The compound may also be formulated for inhalation. In yet another embodiment, the compound may be simply dissolved or suspended in sterile water.

The formulations may conveniently be presented in unit dosage form and may be prepared by any methods well known in the art of pharmacy. The amount of active ingredient which can be combined with a carrier material to produce a single dosage form will vary depending upon the host being treated, and the particular mode of administration. The amount of active ingredient that can be combined with a carrier material to produce a single dosage form will generally be the amount of the compound which produces a therapeutic effect. Generally, out of one hundred percent, this amount will range from about 1 percent to about ninety-nine percent of active ingredients, preferably from about 5 percent to about 70 percent, most preferably from about 10 percent to about 30 percent.

Methods of preparing these formulations or compositions include the step of bringing into association an active compound, such as the compounds of the present invention with the carrier and, optionally, one or more accessory ingredients. In general, the formulations are prepared by uniformly and intimately bringing into association a compound of the present disclosure with liquid carriers or finely divided solid carriers or both and then, if necessary, shaping the product.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound and, if desired, another type of therapeutic agent being administered with the compound of the disclosure. A larger total dose can be delivered by multiple administrations of the agent. Methods to determine efficacy and dosage are known to those skilled in the art.

In general, a suitable daily dose of an active compound used in the compositions and methods of the disclosure will be that amount of the compound that is the lowest dose effective to produce a therapeutic effect. Such an effective dose will generally depend upon the factors described above.

As anticipated above, the compounds may be administered by any appropriate route, for example orally, parenterally, topically, or rectally. It will be appreciated that the preferred route may vary with, for example, the condition of the recipient of the compound and the cancer to be treated. In certain embodiments, the extract may be especially suitable for the preparation of pharmaceuticals for intravenous administration, such as intravenous injection or infusion, provided that it does not contain components with serum-precipitating and/or haemagglutinating properties which disturb such an application. The extract may therefore be provided in the form of ampoule preparations which are directed to intravenous administration. In still other embodiments, the method comprises systemic administration of a subject composition to a subject.

Also provided are methods of treating cancer, which optionally include administering the compounds in conjunction with a chemotherapeutic agent to a subject. Conjunctive therapy includes sequential, simultaneous and separate, or co-administration of the compound and the chemotherapeutic agent in a way that the therapeutic effect of the chemotherapeutic agent is not entirely disappeared when the compound is administered. In certain embodiments, compound and the chemotherapeutic agent may be compounded together in the same unitary pharmaceutical composition including both entities. Alternatively, the combination of compound and chemotherapeutic agent may be administered separately in separate pharmaceutical compositions, each including one of the compound and chemotherapeutic agent in a sequential manner wherein, for example, the compound or the chemotherapeutic agent is administered first and the other second. A chemotherapeutic agent comprises any treatment applicable for cancer treatment.

Exemplary doses of the compounds in the range from about 0.001, 0.01, 0.1, 0.5, 1, 10, 15, 20, 25, 50, 100, 200, 300, 400, 500, 600, or 750 to about 1000 mg/day per kg body weight of the subject. In certain embodiments, the dose of the compound will typically be in the range of about 100 mg/day to about 1000 mg/day per kg body weight of the subject, specifically in the range of about 200 mg/day to about 750 mg/day per kg, and more specifically in the range of about 250 mg/day to about 500 mg/day per kg. In an embodiment, the dose is in the range of about 50 mg/day to about 250 mg/day per kg. In a further embodiment, the dose in the range of about 100 mg/day to about 200 mg/day per kg. In an embodiment, the dose is in the range of about 15 mg/day to 60 mg/day per kg. In a further embodiment, the dose is in the range of about 20 mg/day to 50 mg/day per kg. In an additional embodiment, the dose is in the range of about 25 mg/day to 45 mg/day per kg.

If desired, the effective daily dose of the active compound may be administered as one, two, three, four, five, six or more sub-doses administered separately at appropriate intervals throughout the day, optionally, in unit dosage forms. In yet another embodiment of the present disclosure, the active compound may be administered two or three times daily. In preferred embodiments, the active compound will be administered once daily.

The combined use of either compound (I) or (II) and other chemotherapeutic agents may reduce the required dosage for any individual component because the onset and duration of effect of the different components may be complementary. In such combination therapies, the different active agents may be delivered together or separately, and simultaneously or at different times within the day.

The patient receiving this treatment is any animal in need, including primates, in particular humans.

Wetting agents, emulsifiers, and lubricants, such as sodium lauryl sulfate and magnesium stearate, as well as coloring agents, coating agents, release agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol and the like; and (3) metal-chelating agents, such as citric acid, ethylenediamine tetraacetic acid (EDTA), sorbitol, tartaric acid, phosphoric acid and the like.

In certain embodiments, the compounds of the present invention can be formulated and administered in a prodrug form. A prodrug is generally defined as a biologically inactive derivative of a parent drug molecule, which is activated by a chemical or enzymatic transformation within the body, which, in the process, involves the release of the active drug. In general, prodrugs comprise functional derivatives of the claimed compounds, which are capable of being enzymatically activated or converted into the more active parent form. Thus, in the treatment methods of the present disclosure, the term “administering” encompasses the treatment of the various disorders described with the compound specifically disclosed or with a compound which may not be specifically disclosed, but which converts to the specified compound in vivo after administration to the patient. Conventional procedures for the selection and preparation of suitable prodrug derivatives are described [6].

The the compounds of the present invention may be obtained from commercial sources or can be prepared by the conventional methods known in the art [7, 8] reveals that the BRITE compound collection, consisting of a library of more than 350,000 compounds generated by combinatorial chemistry synthetic routes, was a gift from Biogen Idec. Formula (II) was purchased from Med Chem Express.

The present disclosure now being generally described, it will be more readily understood by reference to the following examples, which are included merely for purposes of illustration of certain aspects and embodiments of the present disclosure and are not intended to limit the present disclosure in any way.

As is evident from the foregoing description, certain aspects of the present disclosure are not limited by the particular details of the examples illustrated herein, and it is therefore contemplated that other modifications and applications, or equivalents thereof, will occur to those skilled in the art. It is accordingly intended that the claims shall cover all such modifications and applications that do not depart from the spirit and scope of the present disclosure.

Moreover, unless defined otherwise, all technical and scientific terms used herein have the same meaning as commonly understood by one of ordinary skill in the art to which the disclosure belongs. Although any methods and materials similar to or equivalent to or those described herein can be used in the practice or testing of the present disclosure, the preferred methods and materials are described above.

### PREFERRED EMBODIMENTS

Embodiment 1. A method of treating or preventing cancer in a subject in need thereof, comprising:
administering to the subject:
(a) a therapeutically effective amount of a compound of Formula (I):

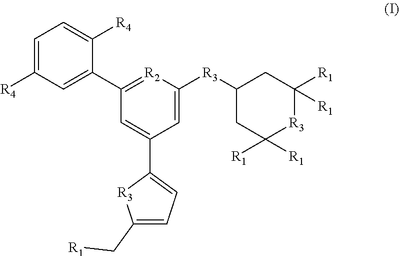

wherein R₁ is independently selected from the group consisting of:
(I) hydrogen
(II) halogen
(III) alkyl or cycloalkyl;
(IV) alkenyl or cycloalkenyl;
(V) alkynyl or cycloalkynyl;
(VI) haloalkyl;
(VII) alkoxy;
(VIII) alkylamino; or
wherein R₂ is independently selected from the group consisting of:
(IX) a —N;
(X) a —P;
(XI) a —CR, wherein R is selected from the group comprising a hydrogen, an alkyl, a cycloalkyl, a haloalkyl, an alkylamino, or an alkoxy; or
wherein R₃ is independently selected from the group consisting of:
(XII) an —O;
(XIII) a —S;
(XIV) a —C(R)₂, wherein R is selected from the group comprising a hydrogen, an alkyl, a cycloalkyl, a haloalkyl, an alkylamino, and/or an alkoxy;
(XV) a —NR, wherein R is selected from the group comprising a hydrogen, an alkyl, a cycloalkyl, or a haloalkyl;
(XVI) a —PR, wherein R is selected from the group comprising a hydrogen, a halogen, an alkyl, a cycloalkyl, or a haloalkyl; or
wherein R₄ is independently selected from the group consisting of:
(XVII) hydrogen;
(XVIII) halogen
(XIX) alkyl or cycloalkyl;
(XX) alkenyl or cycloalkenyl;
(XXI) alkynyl or cycloalkynyl;
(XXII) haloalkyl;
(XXIII) aryl;
(XXIV) cyano;
(XXV) amino;
(XXVI) a —COOR, wherein R is a hydrogen or an alkyl;
(XXVII) a —OCOR, wherein R is an alkyl group;
(XXVIII) acyl;
(XXIX) sulfonyl;
(XXX) a —CON(R)₂, wherein R is selected from the group comprising a hydrogen and/or an alkyl;
(XXXI) a —CONHR, wherein R is selected from the group comprising a hydrogen or an alkyl;
(XXXII) a —PO(OR)₂, wherein R is selected from the group comprising a hydrogen and/or an alkyl;
(XXXIII) an —OR, wherein R is selected from the group comprising a hydrogen, an alkyl, a cycloalkyl, or a haloalkyl;
(XXXIV) an —N(R)₂, wherein R is selected from the group comprising a hydrogen, an alkyl, a cycloalkyl, and/or a haloalkyl;
(XXXV) a —SR, wherein R is selected from the group comprising a hydrogen, an alkyl, a cycloalkyl, or a haloalkyl;
(XXXVI) a nitro;
(XXXVII) a phosphoryl; or
a pharmaceutically acceptable salt, co-crystal, polymorph, hydrate, solvate, prodrug, N-oxide, bioisostere, or a stereoisomer thereof, or
(b) a therapeutically effective amount of a composition comprising a therapeutically effective amount of the compound of Formula (II)

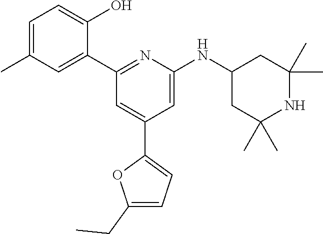

or a pharmaceutically acceptable salt, co-crystal, polymorph, hydrate, solvate, prodrug, or a stereoisomer thereof.

Embodiment 2. The method of embodiment 1, wherein the compound is of Formula (II):

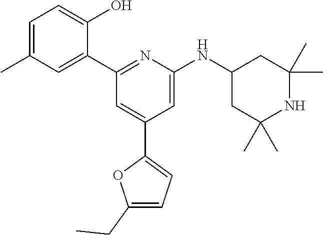

or
(a) or a pharmaceutically acceptable salt, co-crystal, polymorph, hydrate, solvate, prodrug, N-oxide, bioisostere, or a stereoisomer thereof, or
(b) a therapeutically effective amount of a composition comprising a therapeutically effective amount of the compound of Formula (II), or a pharmaceutically acceptable salt, co-crystal, polymorph, hydrate, solvate, prodrug, or a stereoisomer thereof.

Embodiment 3. The method of any of the preceding embodiments, wherein the subject is a mammal.

Embodiment 4. The method of embodiment 3, wherein the mammal is a human.

Embodiment 5. The method of any of the preceding embodiments, wherein a composition comprising an effective amount of compound (I) and one or more pharmaceutical excipients and/or carriers is administered to a subject in need thereof.

Embodiment 6. The method of embodiment 2, wherein a composition comprising an effective amount of compound (II) and one or more pharmaceutical excipients and/or carriers is administered to a subject in need thereof.

Embodiment 7. The method of embodiment 4, wherein the composition is used for treating cancer.

Embodiment 8. The method of embodiment 6, wherein the cancer is breast cancer.

Embodiment 9. The method of any of the preceding embodiments, optionally comprising administering one or more additional therapeutic agents, wherein the additional therapeutic agents are administered prior to, concurrently with, or subsequently to the administration of the compound of Formula (I) or the composition comprising the compound of Formula (I).

Embodiment 10. The method of any of the preceding embodiments, optionally comprising administering one or more additional therapeutic agents, wherein the additional therapeutic agents are administered prior to, concurrently with, or subsequently to the administration of the compound of Formula (II) or the composition comprising the compound of Formula (II).

Embodiment 11. The method of embodiment 6, wherein the treatment kills at least 70% of cancer cells.

Embodiment 12. The method of embodiment 5, wherein the composition is used for treating cancer.

### EXAMPLES

### Example 1

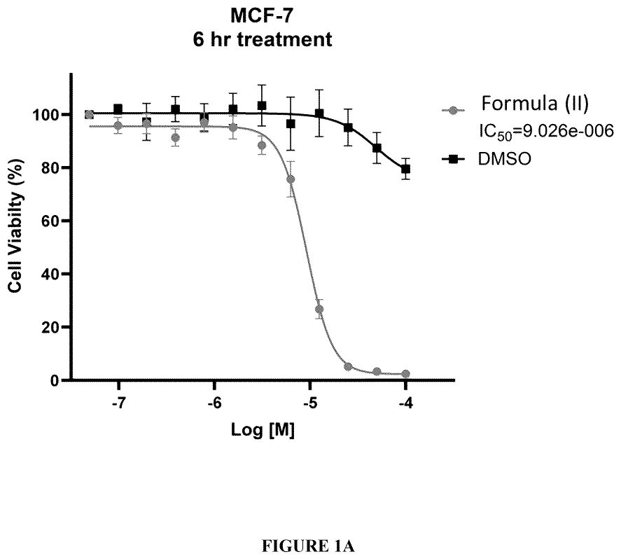

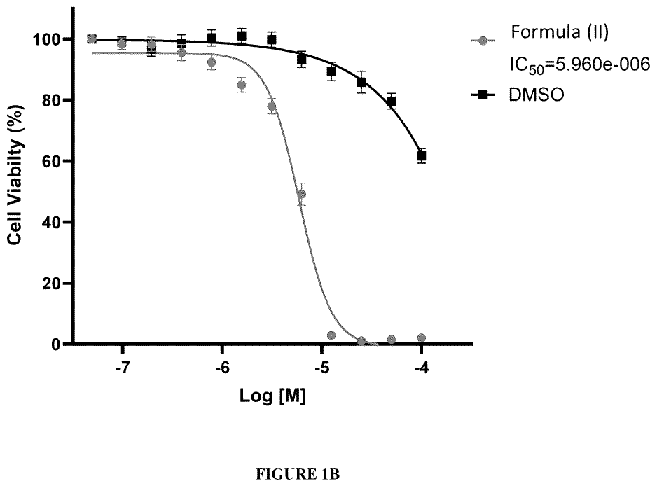

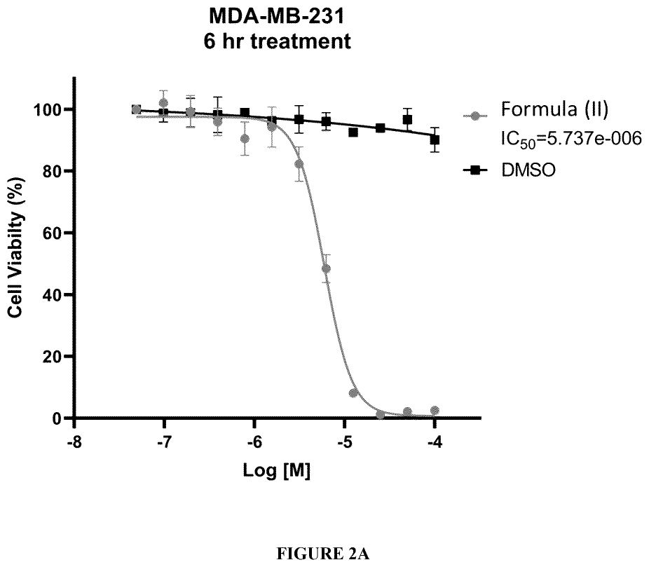

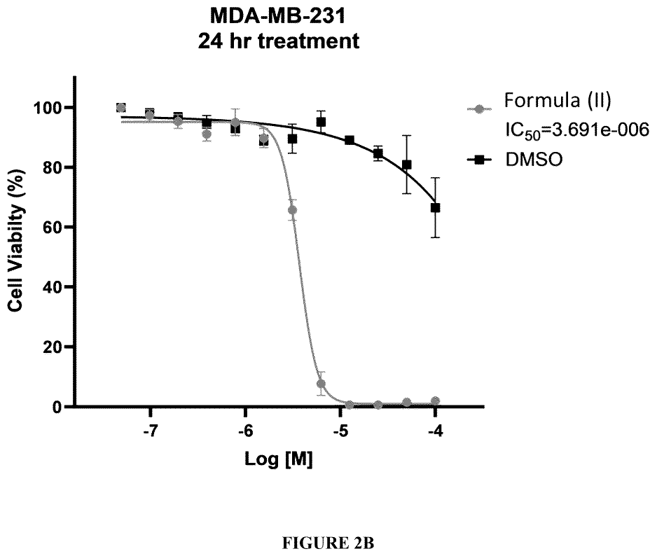

The ability of the compound of Formula (II) to inhibit breast cancer cell growth and/or viability was analyzed based on two breast cancer cell lines: MCF-7 (estrogen and EFG-dependent) and MDA-MB-231 (more aggressive, hormone-independent). It showed significant inhibition in both cell lines following 6 hours and 24-hour treatment periods. In addition, after the application of the said compound, the breast cancer cell growth and/or viability was inhibited with IC₅₀ranging from 3.69±0.46 to 9.14±0.29 μM depending on the cell line used and the time of treatment (6h and 24h treatments). Cancer cell viability in the presence or absence of the compound was measured in both the cell lines. FIGS. 1A and 1B illustrate that after application of the compound, there was sharp decrease in cell viability as compared to the control (DMSO) after treatment for 6 hours and 24 hours, as measured against the MCF-7 cell line. Similarly, FIGS. 2A and 2B shows a sharp decrease in cancer cell viability and a reduction in IC₅₀ values after 24 hours of treatment that in the MDA-MB-21 cell line.

TABLE 1
Inhibitory effect of the compound of Formula
(II) against two different cancer cell lines.

IC50 values (μM)

| Cell Lines | 6-hour treatment | 24-hour treatment |
|---|---|---|
| MCF7 | 9.14 ± 0.29 | 5.90 ± 0.29 |
| MDA-MB-231 | 5.97 ± 0.47 | 3.69 ± 0.46 |

Knowing that the IC₅₀ of cisplatin in MCF7 cells is 5.75±0.02 μM after 24 hours [9] the compound of Formula (II) can be considered to be equally effective in breast cancer treatment because it has comparable IC₅₀ values to that of cisplatin (5.90±0.29 μM after 24 hours). In fact, at values higher than IC₅₀ (i.e. 10 μM), the compound can kill almost 96-99.7% of cancer cells (FIGS. 1A and 1B), while cisplatin kills around only 70% of the cancer cells [9].

### Colorimetric MTT Assay

Formula (II) was purchased from Med ChemExpress and dissolved in DMSO to a final concentration of 10 mM. Cytotoxicity assay was performed on MCF-7 and MDA-MB-231 cell lines. They were treated with 0.1-100 μM of Formula (II) with respective DMSO concentration of 0.002-2%. Please see below the details of the “Cell lines and culture conditions” as well as the “Cytotoxicity Assay”. In brief, in a 96-well plate, cells were seeded along with different concentrations of Formula (II) for 6 and 24 hrs. Cell viability was assessed using a colorimetric MTT assay. Following treatment, MTT was added and the absorbance was measured using a microplate reader. To determine the IC50, data were analysed using nonlinear regression “Dose-Response—Inhibition” curve with the GraphPad prism software.

### Cell Lines and Culture Conditions

MCF-7 and MDA-MB-231, human breast cancer cell lines, were used. The MCF-7 cells were cultured in Dulbecco's modified eagle medium (DMEM) supplemented with 10 g/mL insulin. MDA-MB-231 was maintained in DMEM (Sigma, USA). All media were supplemented with 10% (v/v) FBS, penicillin (100 I.U/mL) and streptomycin (100 ng/mL). The cells were cultured at 37° C. in a humidified incubator with 5% CO₂. Cells were sub-cultured to a fresh media as they reached 80% confluency.

### Cytotoxicity Assay

Proliferation Inhibition Assay (Cytotoxicity). Formula (II) was purchased from Med Chem Express and dissolved in DMSO to a final concentration of 10 mM. Cytotoxicity assay was performed on MCF-7 and MDA-MB-231 cell lines. Briefly, 10⁴ cells/well were seeded in a flat-bottom 96-well plate and were allowed 24 hours to adhere properly. Formula (II) was added at concentrations ranging from 10⁻⁴ to 10⁻⁷ μM with respective DMSO concentration of 2-0.002%, and plates were incubated up to 48 or 72 hours. MTT cell proliferation solution (0.2 mg/mL) was added and incubated for 4 hours. Subsequently, MTT solution was removed, and DMSO was then added. The absorbance was read at 570 nm with reference wavelength at 670 nm using ELISA plate reader (TECAN infinite M200 Pro).

All experiments were repeated at least three times, and results were expressed as the mean±Standard Error of the Mean (SEM). All statistical analyses were performed using GraphPad Prism 8 software (GraphPad Software, San Diego, CA). Percent of cell viability was plotted against log of concentration. Non-linear regression with a variable-slope sigmoidal dose-response curve plot was employed to show the percentage of viable breast cancer cells determined by MTT assay after treatment and to estimate the value of IC₅₀ (half maximal inhibitory concentration).

### Toxicity Study

The toxicity profile analysis was done using an online prediction tool (https://tox.charite.de/protox3/). The lethal dose 50 (LD₅₀) is a standard measure used in toxicology to assess the acute toxicity of a substance. The LD₅₀ value is the dose at which 50% of the test population die due to exposure to the substance. A larger LD₅₀ means the drug has a lower toxicity profile. Based on the prediction of the online tool found in tox.charite.de/protox3, the LD₅₀ of the Formula (II) was 140 mg/Kg, while, for example, imatinib (an FDA-approved drug used to treat cancer), has a smaller LD₅₀ of 100 mg/Kg. This infers that per kg of patient, it takes 140 mg/Kg of Formula (II) before it is toxic per Kg of subject in the test population. On the other hand, it takes less amount of imatinib (100 mg/Kg) before it is toxic per Kg of subject in the test population. Therefore, the compound of Formula (II) is safer (in this respect) compared to imatinib.

### REFERENCES

[1] Global Burden of Disease Cancer Collaboration, Fitzmaurice et al. Global, Regional, and National Cancer Incidence, Mortality, Years of Life Lost, Years Lived With Disability, and Disability-Adjusted Life-Years for 29 Cancer Groups, 1990 to 2017: A Systematic Analysis for the Global Burden of Disease Study [published correction appears in JAMA Oncol. 2020 Mar. 1; 6(3):444][published correction appears in JAMA Oncol 2020 May 1;6(5):789][published correction appears in JAMA Oncol. 2021 Mar. 1:7(3):466]. JAMA Oncol. 2019; 5(12):1749-1768. doi:10.1001/jarnaoncoL2019.2996,
[2] Sung et al., Global Cancer Statistics 2020: GLOBOCAN Estimates of Incidence and Mortality Worldwide for 36 Cancers in 185 Countries, CA Cancer J Clin. 2021; 71: 209.
[3] Siegel et al., Cancer statistics, 2019, CA Cancer J Clin. 2019; 69: 7.
[4] Romani et al., Cisplatin in cancer treatment. Biochemical Pharmacology. 2022; 206: 115323; Ghosh S, Cisplatin: The first metal based anticancer drug. Bioorganic Chemistry. 2019; 88: 102925
[5] Duarte et al., New Trends for Antimalarial Drugs: Synergism between Antineoplastics and Antimalarials on Breast Cancer Cells. Biomolecules, 2020, 10(12): 1263.
[6] Wihnan et al., 14 Biochem. Soc. Trans. 375-82 (1986); Stella et al., Prodrugs: A Chemical Approach to Targeted Drug Delivery in Directed Drug Delivery, 247-67 (1985).
[7] Wigle et al., Inhibitors of RecA Activity Discovered by High-Throughput Screening: Cell-Permeable Small Molecules Attenuate the SOS Response in Escherichia coli. Journal of Biomolecular Screening 14(9); 2009.
[8] Sexton et al., Novel Inhibitors of E. coli RecA ATPase Activity. Current Chemical Genomics, 2010, 4, 34-42.
[9] Suberu J O et al., Comparative cytotoxicity of artemisinin and cisplatin and their interactions with chlorogenic acids in MCF7 breast cancer cells. Chem Med Chem. 2014; 9(12):2791-7

1. A method of treating breast cancer in a subject in need thereof, comprising:
administering to the subject:
(a) a therapeutically effective amount of a compound of Formula (I): 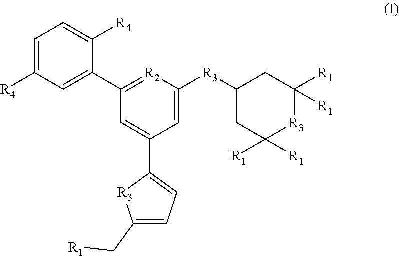
wherein R₁ is independently selected from the group consisting of:
hydrogen;
halogen;
alkyl or cycloalkyl;
alkenyl or cycloalkenyl;
alkynyl or cycloalkynyl;
haloalkyl;
alkoxy; and
alkylamino;
wherein R₂ is N or P;
wherein R₃ is independently selected from the group consisting of:
an —O;
a —S;
a —NR, wherein R is selected from the group consisting of: a hydrogen, an alkyl, a cycloalkyl, and a haloalkyl; and
a —PR, wherein R is selected from the group consisting of: a hydrogen, a halogen, an alkyl, a cycloalkyl, and a haloalkyl;
wherein R₄ is independently selected from the group consisting of:
hydrogen;
halogen;
alkyl or cycloalkyl;
alkenyl or cycloalkenyl;
alkynyl or cycloalkynyl;
haloalkyl;
aryl;
cyano;
amino;
acyl;
sulfonyl;
a —PO(OR)₂, wherein R is selected from the group comprising consisting of: a hydrogen and an alkyl;
an —OR, wherein R is selected from the group comprising consisting of: a hydrogen, an alkyl, a cycloalkyl, and a haloalkyl;
an —N(R)₂, wherein R is selected from the group comprising consisting of: a hydrogen, an alkyl, a cycloalkyl, and a haloalkyl;
a —SR, wherein R is selected from the group comprising consisting of: a hydrogen, an alkyl, a cycloalkyl, and a haloalkyl;
a nitro; and
a phosphoryl;
or a pharmaceutically acceptable salt, co-crystal, hydrate, solvate, or a stereoisomer thereof, or
(b) a therapeutically effective amount of a composition comprising a therapeutically effective amount of the compound of Formula (I), or a pharmaceutically acceptable salt, co-crystal, hydrate, solvate, or a stereoisomer thereof.

2. A method of treating breast cancer in a subject in need thereof, comprising:
administering to the subject:
(a) a therapeutically effective amount of a compound of Formula (II): 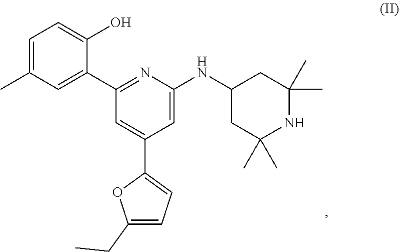
or a pharmaceutically acceptable salt, co-crystal, hydrate, solvate, or a stereoisomer thereof, or
(b) a therapeutically effective amount of a composition comprising a therapeutically effective amount of the compound of Formula (II), or a pharmaceutically acceptable salt, co-crystal, hydrate, solvate, or a stereoisomer thereof.

3. The method of claim 1, wherein the subject is a mammal.

4. The method of claim 3, wherein the mammal is a human.

5. The method of claim 1, wherein a composition comprising an effective amount of compound (I) and one or more pharmaceutical excipients and/or carriers is administered to a subject in need thereof.

6. The method of claim 2, wherein a composition comprising an effective amount of compound (II) and one or more pharmaceutical excipients and/or carriers is administered to a subject in need thereof.

7-9. (canceled)

10. The method of claim 2, further comprising administering one or more additional therapeutic agents, wherein the additional therapeutic agents are administered prior to, concurrently with, or subsequently to the administration of the compound of Formula (II) or the composition comprising the compound of Formula (II).

11. The method of claim 6, wherein the treatment kills at least 70% of cancer cells.

12-15. (canceled)

16. The method of claim 1, further comprising administering one or more additional therapeutic agents, wherein the additional therapeutic agents are administered prior to, concurrently with, or subsequently to the administration of the compound of Formula (I) or the composition comprising the compound of Formula (I).
